(19) United States
(12) Patent Application Publication
(10) Pub. No.: US 2004/0081663 A1
(43) Pub. Date: Apr. 29, 2004

(54) Pharmaceutical composition for treatment and prevention of cancer and the preparation thereof

(76) Inventors: Chun-Min Chang, Hsinchu (TW); Ming-Da Hu, Taipei (TW); Chien-Yang Li, Taipei (TW); Chih-Chung Yeh, Taipei (TW)

Correspondence Address: BIRCH STEWART KOLASCH & BIRCH, PO BOX 747, FALLS CHURCH, VA, 22040-0747, US

(21) Appl. No.: 10/443,801

(22) Filed: May 23, 2003

(30) Foreign Application Priority Data
Oct. 25, 2002 (TW) 091125231

(51) Int. Cl. A61K 35/70; A61K 35/72

(52) U.S. Cl. 424/195.16

(57) ABSTRACT

A composition for treatment and prevention of cancer including a Monascus fermentation extract as active component. The inventive Monascus extract is produced by liquid fermentation, and includes at least one component with three characteristic ultraviolet absorption peaks at 230, 237 and 246 nm.

### BACKGROUND OF THE INVENTION

1. Field of the Invention

The present invention relates to a composition for treatment and prevention of cancer and in particular to a Monascus sp. fermented Monascus composition for preventing or treating cancer and fabrication method thereof.

2. Description of the Related Art

Red rice, known in Chinese as Hongqu (rendered herein in accordance with pinyin standards of Chinese Romanization) has been known and used for hundreds of years in China in rice wine making and as a food preservative. Red rice is known mostly for its use in food as a preservative and colorant, and its uses in the dye industry. In addition, red rice has been known as an ancient Chinese medicine or an ingredient in certain ancient Chinese prescriptions.

Traditionally, red rice is a fermentation product of rice fermenting with Monascus sp., which is still used in Taiwan, mainland China, and Asian communities in North America for wine making and food coloring. In Ben Cao Gang Mu (rendered herein in accordance with pinyin standards of Chinese Romanization), a medical work published during the Ming dynasty (1368-1644), red rice is described as mild, nonpoisonous, and useful for treating indigestion and diarrhea. Red rice is also described as useful for improving blood circulation and promoting the health of the spleen and stomach.

Mao et al. (U.S. Pat. No. 6,046,022 and WO Pub. No. 98/14177) teach compositions that comprise red yeast fermentation products, which can be used as natural dietary supplements and/or medicaments for the treatment or prevention of hyperlipidemia and associated disorders and symptoms, such as cardiovascular diseases, cerebrovascular diseases, diabetes, hypertension, obesity, asthenic breathing, chronic headache, chest pain and tightness, limb swelling and distention, loss of appetite and excess expectoration.

The red and yellow pigments of Monascus purpureus, such as monascorubin and monascin, have been purified by Fielding et al. in 1961 (see J. Chem. Soc, 4579-4589). The culture conditions and its effect on pigmentation of Monascus purpureus have also been studied (see Broder et al., 1980, J Food Sci, 45:567-469). The antibacterial activity of Monascus purpureus extract, especially against Bacillus species, is also detected and disclosed by Wong (Plant Physiol, 60:578-581, 1977).

Mao et al. also disclose (U.S. Pat. No. 6,046,022) several Monascus sp. producing lovastatin, an inhibitor for HMG-CoA reductase that is a key enzyme for cholesterol synthesis, by specific fermentation. Thus, the fermentation products obtained are effective for inhibiting cholesterol synthesis and reducing hyperlipidemia accordingly.

In addition, some prior arts disclose lovastatin as being effective for treatment and prevention of cancer. Hawk et al. disclose lovastatin inhibiting the formation of lung epithelial cells in mice and also growth of NNK-induced lung tumors therein. Lovastatin is also found to be effective for inhibition of various cancer cells, see Cancer Letters 137, 1999, 167-172, Girgert et al.; Clinical Cancer research Vol. 7, 158-167, January 2001, Dimitroulakos et al; and Acta Neuropathol, 2001, 101:217-224, Schmidtet et al.

Mayers et al. teach a method of treating prostatic adenocarcinoma with lovastatin (see U.S. Pat. No. 6,040,334). Sebti et al. teach a method of blocking aberrant Ras signaling in a mammal while avoiding excessive cell toxicity by administration of lovastatin and geranylgeraniol (see U.S. Pat. No. 6,083,979).

### SUMMARY OF THE INVENTION

The present invention is based on the discovery that Monascus extracts is effective for treatment and prevention of cancer. Thus, a composition for treatment and prevention of cancer and a method for fabrication thereof are provided accordingly. The terms “red rice”, “Monascus extract” or “Monascus composition” discussed hereinbelow and the claimed invention refer to a fermentation product fermented with at least one Monascus strain.

The composition for treatment and prevention of cancer according to the present invention comprises Monascus compositions as the active components, and is effective for treating lung and breast cancers. The Monascus composition can be a solid or liquid fermentation product of a Monascus strain.

In a preferred embodiment, the Monascus composition comprises at least one component with three characteristic ultraviolet absorption peaks at 230, 237 and 246 nm.

The present invention further provides a pharmaceutical composition for treatment and prevention of cancer comprising a sufficient amount of Monascus extract characterized by three ultraviolet absorption peaks at 230, 237 and 246 nm, and a pharmaceutical carrier or excipient.

In a preferred embodiment, the Monascus composition has the characteristics of retention times of 6.6, 7.7 and 8.7 min. on HPLC using a Cosmosil 5C18-ARII column (4.6.times.250 mm) and elution with methanol-water-H₃PO₄ (3.2 L: 0.8 L: 1.6 ml) at a flow rate of 1 ml/min and detected with 237 nm. The preferred Monascus composition comprises lovastatin.

A method for fabricating Monascus composition is also provided according to the present invention. A liquid culture medium is provided of grain/starch:peptone:MgSO₄.7H₂O:water=1-7 g: 0.5-3 g: 0.1-1 g: 100 g. A Monascus strain is cultured in the liquid culture medium at a temperature of about 20 to 35° C., an agitation rate of 100 to 400 rpm, and an aeration (volume of air per volume of liquid per minute) rate of 0.1 to 1 vvm for 4 to about 12 days. After culturing, the culture medium is filtered to obtain a Monascus extract as the Monascus composition.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a visible light spectrum of a Monascus extract for treatment and prevention of cancer according to one embodiment of the present invention;

FIG. 2 is a HPLC profile of a Monascus extract for treatment and prevention of cancer according to one embodiment of the present invention;

FIGS. 3A and 3B are UV spectrums of a Monascus extract for treatment and prevention of cancer according to one embodiment of the present invention;

FIG. 4 is a HPLC profile of a liquid cultured Monascus extract according to one embodiment of the present invention;

FIG. 5 is a chart illustrating the in vivo effects of Monascus compositions on tumor prevention and comparison with the effects of lovastatin in one embodiment of the present invention;

FIG. 6 is a chart illustrating the in vivo effects of Monascus compositions on tumor suppression and comparison with the effects of lovastatin in one embodiment of the present invention;

FIG. 7 is a chart illustrating the in vitro suppression effects of Monascus compositions on lung cancer and comparison with the effects of lovastatin in one embodiment of the present invention; and

FIG. 8 is a chart illustrating the in vitro suppression effects of Monascus compositions on breast cancer and comparison with the effects of lovastatin in one embodiment of the present invention.

### DETAILED DESCRIPTION OF THE INVENTION

Preferred embodiments are disclosed below, showing media and process for producing Monascus compositions for treatment and prevention of cancer according to the present invention.

### Culture Media for Monascus sp.

### Culture Media for Solid Fermentation

The anti-cancer Monascus composition according to the invention can be obtained by conventionally solid fermentation. Some culture media for solid fermentation are listed below.

Solid fermentation culture medium I

| | | |
|---|---|---|
| Nonglutinous rice | 100 | g |
| Water | 500 | ml |

Solid fermentation culture medium II

| | | |
|---|---|---|
| Nonglutinous rice | 100 | g |
| Glycerin | 2.5 | ml |
| Water | 500 | ml |

Solid fermentation culture medium III

| | | |
|---|---|---|
| Nonglutinous rice | 100 | g |
| Glycerin | 2.5 | ml |
| Peptone | 1 | g |
| Water | 500 | ml |

Solid fermentation culture medium IV

| | | |
|---|---|---|
| Rice | 60-120 | g |
| Water | 50 | ml |

(Containing 0-10% glycerin, 0-10% glucose, 0-8% peptone, 0-3% malt extract, and 0-0.4% MgSO₄.7H₂O)

Each medium was mixed well, the pH value thereof adjusted to 3-6.5 with acetic acid, and then sterilized at 121 C for 30 minutes. The sterilization time can be extended for larger amounts of medium to ensure thorough sterilization.

### Culture Media for Liquid Fermentation

In addition to conventional solid fermentation of Monascus sp., the present invention further provides deep broth culture media, i.e. liquid media, for liquid fermentation of Monascus sp. to obtain Monascus compositions.

Liquid fermentation culture medium I

| | | |
|---|---|---|
| Glutinous rice | 5 | g |
| Glycerin | 9 | ml |
| Peptone | 1.5 | g |
| Water | 100 | ml |

Liquid fermentation culture medium II

| | | |
|---|---|---|
| Potato starch | 4.5 | g |
| Glycerin | 10 | ml |
| Peptone | 1.5 | g |
| MgSO₄.7H₂O | 0.4 | g |
| Water | 100 | ml |

Liquid fermentation culture medium III

| | | |
|---|---|---|
| Glutinous rice | 5 | g |
| Glycerin | 7 | ml |
| Peptone | 1.5 | g |
| Casein | 3 | g |
| Glucose | 4 | g |
| MgSO₄.7H₂O | 0.4 | g |
| Water | 100 | ml |

Liquid fermentation culture medium IV

| | | |
|---|---|---|
| Grain/starch | 1-7 | g |
| Glycerin | 0-10 | ml |
| Peptone | 0.5-3 | g |
| Malt extract | 0-3 | g |
| Casein | 0-5 | g |
| Glucose | 0-6 | g |
| MgSO₄.7H₂O | 0.1-1 | g |
| Water | 100 | ml |

In the above liquid media, the liquid fermentation culture medium IV illustrates a preferred recipe and concentration ratios for liquid fermentation of Monascus sp. The recipe can be adjusted according to the individual Monascus strain cultured.

The media are sterilized at 121 C for 30 minutes with agitation. The sterilization time can be extended depending on the amount of medium to ensure thorough sterilization.

### Source of Monascus sp.

Many species of Monascus fungi are easily available or can be ordered by the public from many international microorganism institutes, such as American Type Culture Center (ATCC) in the United States or Food Industry Research and Development Institute in Taiwan assigned with “CCRC” prefix.

Some Monascus species that can be obtained by the public are listed below with corresponding ATCC or CCRC number.

| | | |
|---|---|---|
| Monascus | ATCC | 6405, 16360, 16365, 16385 |
| purpureus Went | | 16427, 26264, 34892, 36113 |
| | | 36114, 36928, 48162 |
| | CCRC | 31497, 31498, 31499, 31500 |
| | | 31501, 31504, 31528, 31530 |
| | | 31536, 31540, 31541, 31542 |
| | | 31615, 32668, 32690, 32966 |
| | | 33325, 33327, 33328 |
| Monascus ruber | ATCC | 13692, 15670, 16246, 16366 |
| van Tieghem | | 16378, 18199, 22080, 58358 |
| | CCRC | 31523, 31529, 31532, 31533 |
| | | 31534, 31535, 31538, 31539 |
| | | 33303, 33309, 33314, 33323 |
| | | 33324, 33326, 33329, 33448 |
| Monascus pilosus | ATCC | 16363, 16364, 16368, 16369, 62949 |
| Sato | CCRC | 31502, 31503, 31505, 31526 |
| | | 31527, 31537, |
| Monascus albidus | ATCC | 16357, 16358 |
| Sato | | |
| Monascus | ATCC | 16364 |
| pubigerus Sato | | |
| Monascus sp. | CCRC | 31746, 31747, 32807, 32808, 32809 |
| Monascus | CCRC | 33310 |
| floridanus | | |
| Monascus | CCRC | 33446 |
| sanguineus | | |
| Monascus kaoliang | CCRC | 31506 |
| Monascus albus | CCRC | 33372 |

### Culturing Conditions

### Solid Phase Fermentation

A Monascus strain is added to one of the solid fermentation culture media mentioned above, mixed well, and cultured at 15-35° C. for 7-30 days (20° C. and 20 days is preferred) with occasional tumbling.

### Liquid Phase Fermentation

A Monascus strain is inoculated in a flask loaded with one of the solid phase media disclosed above or a conventional PDA medium. The flask is placed in an orbital shaker and consistently agitated at 150 rpm. The temperature is controlled at 20-35° C. and 25° C. is preferred. The Monascus strain grows in the medium and turns red. The fermented broth is transferred to a fermentor after 2-7 days solid fermentation in the flask. In the fermentor, fermentation temperature is controlled at 15-35° C. (25° C. is preferred) with an agitation rate of 100-400 rpm, (300 rpm is preferred for a 50 L fermentor) and with aeration rate of 0.1-1 vvm (0.5 vvm is preferred). The Monascus is fermented for 4 to 21 days and 14 days is preferred. Generally, most of the liquid medium will be consumed by the Monascus strain during culturing.

### The Extraction of Monascus Composition

### Solid-Liquid Separation

The crude fermentation product from the fermentor is decanted and separated by centrifugation, filtration, or press filter separation and collected. The filtrate is further filtered through a sanitized micropore filter, such as a 0.2 micron filter, to exclude suspended small particles. The derived filtrate is further prepared in sterile conditions to obtain a Monascus composition.

### Extraction process

Solids resulting from the above solid fermentation or the solid-liquid separation are spray-dried or dried in an oven at 50-80° C. The dried products are granulized by a blender if required. The dried powder is extracted subsequently by water or a buffer solution, such as saline or phosphate buffer, in a concentration of 10 to 1 with stirring for 4-15 hours at 4-25° C., preferably 10 hours at 4° C. Organic solvents, such as methanol, ethanol, or propanol, can be added for better extraction. In an embodiment, 50% methanol or ethanol is added for extraction. Finally, the extract is separated from the solid content by centrifugation, filtration, or a press filter.

### Sample Preparation for Treatment and Prevention of Cancer Tests

The above extracts were further filtered through a sanitized micropore filter such as 0.2 micron to exclude suspended small particles. The derived filtrates were further prepared in sterile condition to obtain Monascus compositions for the following anti-cancer tests. For extracts with organic solvents, the organic solvents in the extracts are evaporated or dialyzed against water before the micropore filtration.

### The Analysis of Monascus Compositions

In one embodiment, a Thermo separation Products Spectra System HPLC with an UV6000LP photo-diode-array detector was utilized for the subsequent composition analysis. The column was Cosmosil 5C18-ARII 4.6×250mm. Methanol was added to the above samples at 1 to 1 ratio. Samples were further filtered through a 0.2-0.4 micron filter to exclude suspended particles. The mobile phase for the HPLC analysis was methanol: water: H₃PO₄ at a ratio of 3.2 L: 0.8 L: 1.6 ml. The flow rate was 1 ml/minute. The absorption was detected at 237 nm wave length.

### EXAMPLE 1

Monascus ruber (ATCC 18199) was seeded to a medium comprising 1 Kg nonglutinous rice, 25 ml glycerin, and 500 ml water. The culture was fermented at 20° C. for 25 days in clean condition. A crude fermentation product was obtained when the rice in the medium turned deep brown red. The crude fermentation product was collected and dried in an oven at 80° C.

### EXAMPLE 2

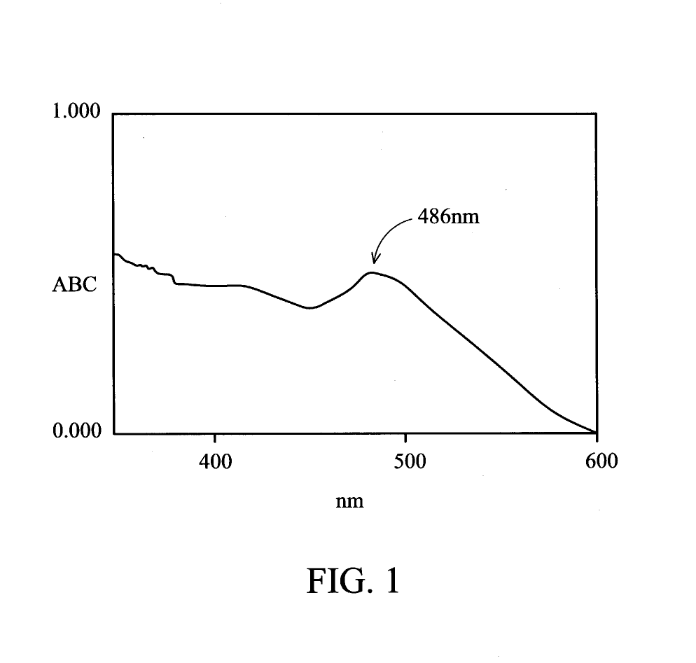

Monascus purpureus (ATCC 48162) was inoculated in a 3 L flask with 300 ml PDA medium. The flask was placed in an orbital shaker and agitated at a rate of 150 rpm for fermentation. The temperature was controlled at 25° C. and the Monascus grew in the medium and turned red. The broth was transferred to a 14 L fermentor after 3 days solid fermentation. The liquid medium loaded in the fermentor was liquid fermentation culture medium III described above. The temperature of the fermentor was controlled at 25° C., the agitation rate was 300 rpm and the aeration rate was 0.5 vvm for 14 days fermentation. The mycelium in the medium was collected on the 26^{th} day and separated by centrifugation. The solid content was dried in an oven at 80° C. The liquid supernatant had a brown red color and was preserved at −20° C. until use. FIG. 1 shows the spectrum of the liquid supernatant scanned with a visible light with absorption peak appearing at about 486 nm.

### EXAMPLE 3

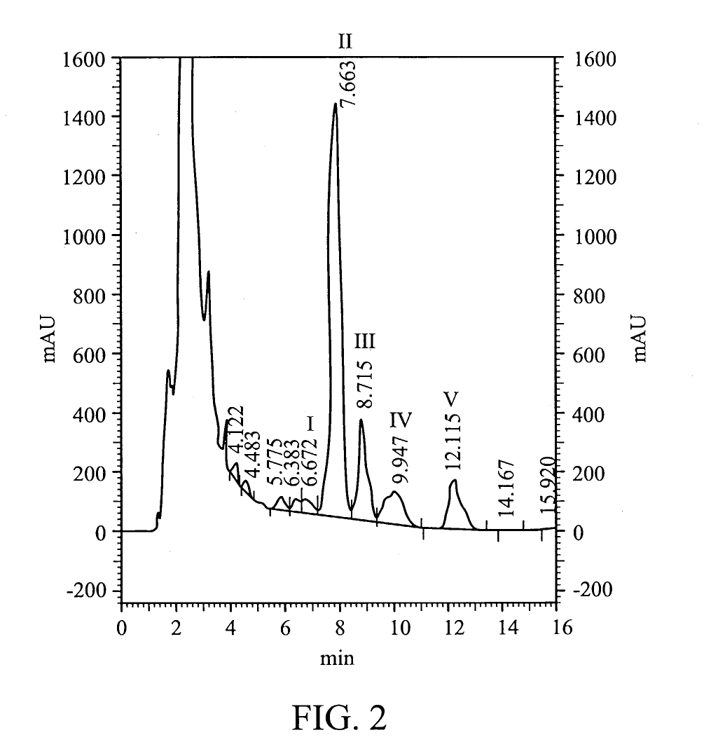

5g of Monascus dried powder obtained from example 2 was extracted with 50 ml of 50% methanol stirred overnight at room temperature. The extract was obtained by 10,000 rpm centrifuging for 10 minutes. The supernatant was collected and analyzed by a Thermo Separation Products Spectra System HPLC with UV6000LP photo-diode-array detector. The column for separation was Cosmosil 5C18-ARII 4.6×250 mm. The sample was pre-filtered through a 0.2 micron filter. Methanol was added to obtain a proper dilution for HPLC analysis. The mobile phase for the HPLC analysis was methanol: water: H₃PO₄ at a ratio of 3.2 L: 0.8 L: 1.6 ml. The flow rate was 1 ml/minute. The absorption was detected at 237 nm. A characteristic HPLC fingerprint profile of the powder in example 2 was obtained, as shown in FIG. 2.

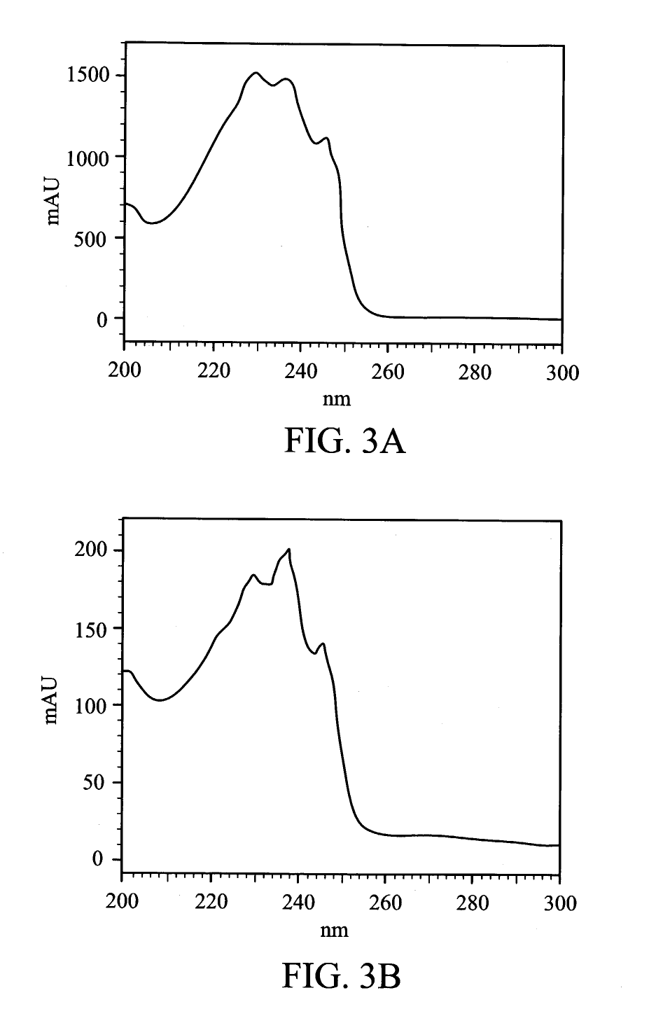

FIG. 2 shows peaks at retention time of about 6.6 (peak I), 7.7 (peak II), 8.7 (peak III), 9.9 (peak IV), and 12.1 (peak V) minutes. The major peak is at the retention time of about 7.7 minutes (peak II), which is identified as the peak of the acid form of lovastatin. The peak III at the retention time of 8.7 minutes is also identified as the lactone form of lovastatin. These components have specific UV absorption profiles. Namely, there are three characteristic UV absorption peaks at 230 nm, 237 nm, and 246 nm as shown in FIGS. 3A and 3B.

### EXAMPLE 4

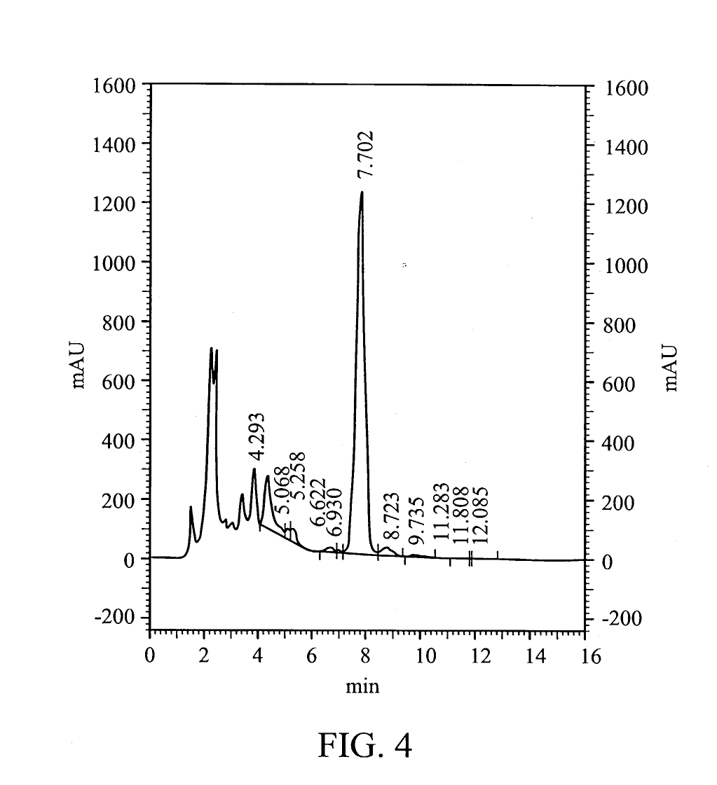

40 ml of the above liquid fermentation broth was centrifuged at 10,000 rpm for 10 minutes to collect the supernatant. The supernatant was subsequently filtered through a 0.2 micron filter. The derived filtrate was further analyzed by HPLC. The HPLC analytical conditions were the same as in example 3. FIG. 4 shows the HPLC profile of the supernatant of sample II. The characteristic peaks of about 6.6, 7.7, 8.7, 9.7, and 12.1 minutes are also observed and the peak of 7.7 minutes shows a stronger response than the other peaks. These fractions also had three characteristic UV absorption peaks at 230 nm, 237 nm, and 246 nm.

### Evaluation of Cancer Treatment Effectiveness

Lewis lung (LLC) peritoneal carcinomatosis model was used for treatment and prevention of cancer in vivo animal tests, referring to Bertram J S, Janik P., Cancer Lett 1980 November;11(1):63-73 ; Hirazumi A, Furusawa E. Phytother Res. 1999 August;13(5):380-7; Itoh H, Noda H, Amano H, Ito H. Anticancer Res. 1995 September-October;15(5B):1937-47; Wallace P K, Morahan P S., J Leukoc Biol. 1994 July;56(1) :41-51.; Redmond HP, Schuchter L, Bartlett D, Kelly C J, Shou J, Leon P, Daly J M. , J Surg Res. 1992 April;52(4):406-11; Kagawa K, Yamashita T, Tsubura E, Yamamura Y., Cancer Res. 1984 February;44(2):665-70; and Yamamoto T, Yamashita T, Tsubura E., Invasion Metastasis. 1981; 1(1) :71-84.

Twenty male C57BL/6J mice of two month old were separated into four groups for 5 mice/each. Each mouse was inoculated with 1×10⁵ LLC1 tumor cells (Lewis lung cancer cell line) by abdominal injection around the thigh on the first day, i.e. day 0. Each group of mice was treated differently for 13 days as described below and treatment was stopped for 12 days. The mice were sacrificed on the 26^{th} day. The abdomen of each mouse was dissected to examine the tumor size. The size of tumor was defined as width times length.

### Control Group

5 mice were used as the control group. The mice in the control group were injected daily with 100 microliters of saline with propylene glycol (3.05 μl propylene glycol in 100 ml normal saline) for 13 days. The treatment was stopped for 12 days and the mice were sacrificed on the 26^{th} day after inoculation with LLC1 tumor cells. All mice had tumors with an average size of 12.38.

### Tumor Resistance Activity of Lovastatin

Authentic lovastatin sample was prepared and dissolved in saline with ethylene glycol as indicated above. The dose amount for administration was adjusted according to mice body weight. The dose amount indicated was the proportional equivalent of lovastatin for human subjects of 60 Kg body weight.

### Lovastatin Equivalent to 80 mg/Day/Person (L80)

Five C57BL/6J mice were inoculated with 1×10⁵ LLC1 cells on day 0 as described above. Lovastatin equivalent of 80 mg/person was injected daily on the other side of the abdomen every day for 13 days. The treatment was stopped for 12 days and the mice were sacrificed on the 26^{th} day after inoculation of LLC1 tumor cells. All mice had tumors with an average size of 9.96.

### Lovastatin Equivalent to 200 mg/Day/Person (L200)

The same procedures of L80 were followed except that the dose amount for administration was changed to 200 mg/day/person equivalent. Three mice had tumors with an average size of 6.8.

### Monascus Composition Tumor Resistance Activity

The liquid fermentation product obtained from sample 2 was centrifuged 10,000 rpm for 20 minutes. The supernatant was collected and further filtrated with a 0.2 micron filter in sterile condition. The concentration of lovastatin in the derived filtrate was detected as 0.45 mg/ml. The dose amount for administration was adjusted according to mice body weight. The equivalent amount of lovastatin for human subjects was used for mice tumor resistance tests as indicated.

### Monascus Composition Equivalent to 80 mg Lovastatin/Day/Person (M80)

The same procedures of example L80 were followed except that the sample was Monascus composition. The dose amount for administration was equivalent to 80 mg lovastatin/day/person. The filtrate was administrated in the ratio of 60 μl filtrate to 40 μl saline. Only two mice had a tumor and the average size of the tumors was 4.8.

### Monascus Composition Equivalent to 200 mg Lovastatin/Day/Person (M200)

The same procedures of example M80 were followed except that the administration sample was Monascus composition. The dose amount for administration was equivalent to 200 mg lovastatin/day/person. The filtrate was administrated in the ratio of 60 μl filtrate to 40 μl saline. Only one mouse had a tumor and the size was 3.25.

### Tumor Resistance Analysis

### Tumor Occurring Percentage

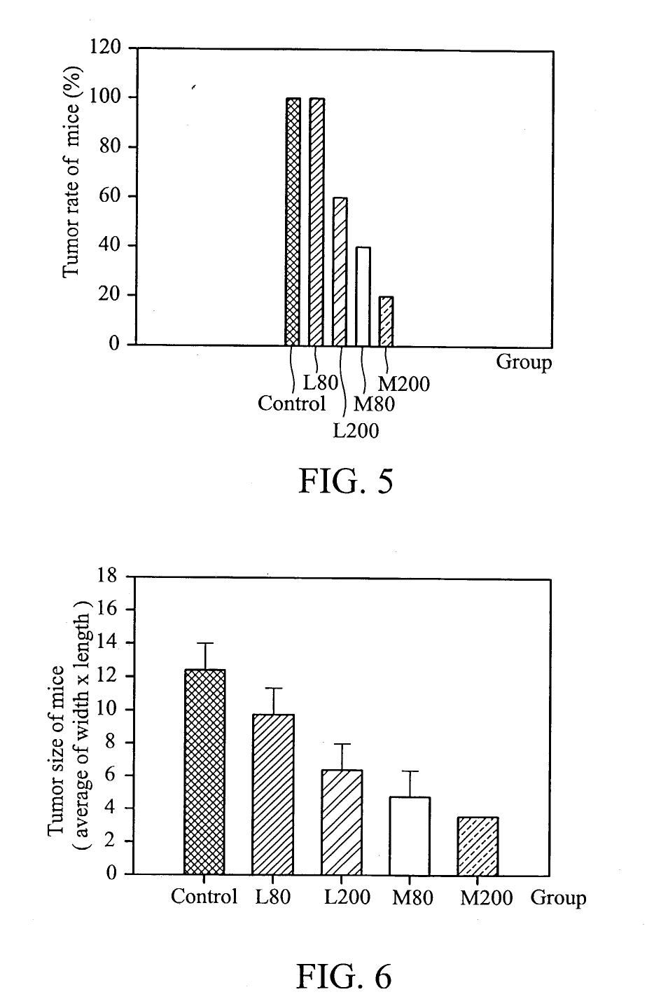

The percentage of mice with tumors receiving different treatment was analyzed and the results are shown in FIG. 5. It is obvious that the groups of mice treated with Monascus compositions, i.e. M80 and M200, had a significantly smaller chance of tumors than the control and lovastatin groups L80 and L200.

### Tumor Size

The tumor size of each group of mice was further analyzed and the results are shown in FIG. 6. Higher doses of lovastatin (L200) and the Monascus compositions M80 and M200 resulted in smaller tumor sizes. The tumor sizes of the mice treated with Monascus compositions was significantly smaller than the mice in the control and lovastatin groups.

### Analysis and Anti-Cancer Activity of Market Samples

Three samples of Monascus fermented red rice were purchased from a local market in Taiwan. The samples were extracted and analyzed as in example 3. However, the HPLC profiles thereof did not show the characteristic absorption peaks. The tumor resistance tests were conducted as in example 8 and example 9. The results were similar to the control group and no tumor resistance activity was observed.

### In Vitro Cancer Cell Suppression Tests

Large lung cancer cell line NCI-H460 and mammary epithelial cell line MCF7 are recommended by National Cancer Institute (NCI) as representative cell lines for in vitro cancer drug studies. The two cell lines were employed for the anti-cancer effects studies of lovastatin and the Monascus compositions.

### Lung Cancer Cell Line NCI-H460

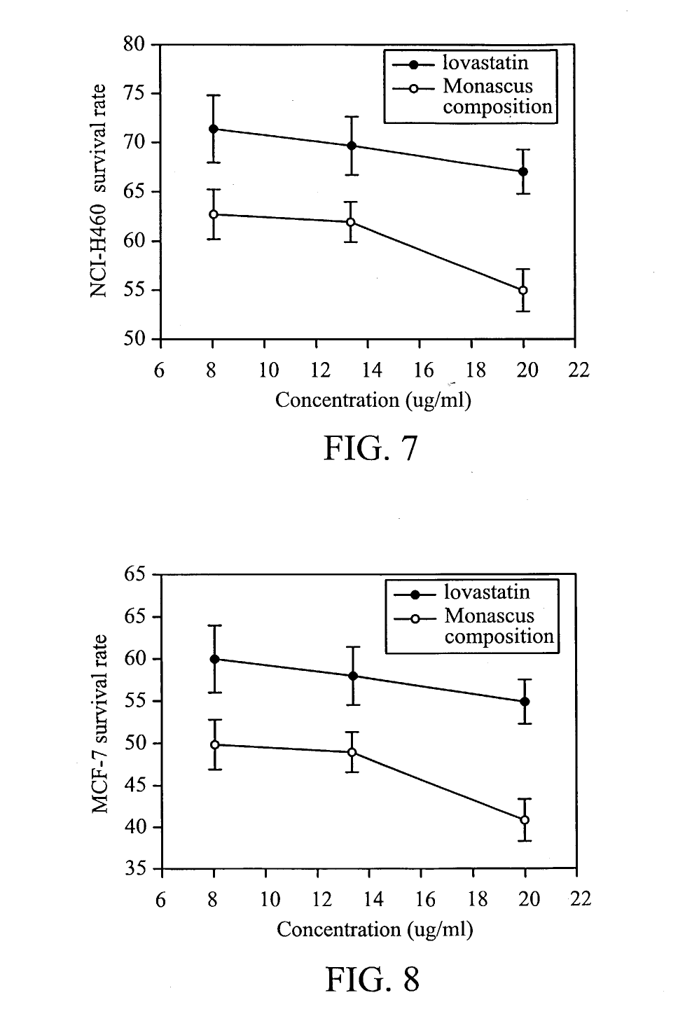

Large lung cancer cell line NCI-H460 was purchased from ATCC, USA, and cultured according to the method recommended by ATCC. The cells were cultured in minimum essential medium Eagle with 2 mM L-glutamine and Earle's BSS adjusted to containing 1.5 g/L sodium bicarbonate, 0.1 mM non-essential amino acids, 1.0 mM sodium pyruvate, 0.01 mg/ml bovine insulin, and 10% fetal bovine serum. The cells were cultured in a cell culture chamber with 5% CO₂, saturated moisture, at 37° C. When the cells were confluent, they were sub-cultured with various concentrations of lovastatin or Monascus compositions under sterile conditions. 4 mg of lovastatin was dissolved in 0.5 ml DMSO and diluted with RPMI 1640 before use. Monascus compositions were adjusted with RPMI 1640 to obtain equivalent concentration of lovastatin in the test sample. To eliminate DMSO effect, the final concentration of DMSO in lovastatin group, Monascus group and the control group were adjusted relative to each other. The cells were further cultured for 1-2 days and the viability thereof was analyzed by MTT method. The Monascus composition had a higher inhibition effect than the lovastatin, as shown in FIG. 7.

### Mammary Epithelial Cell Line MCF7

Mammary epithelial cell line MCF7 was purchased from ATCC, USA, and cultured according to the method recommended by ATCC. The cells were cultured in RPMI 1640 with 2mM L-glutamine adjusted to containing 1.5 g/L sodium bicarbonate, 4.5 g/L glucose, 10 mM HEPES, and 10% fetal bovine serum. The cells were cultured in a cell culture chamber with 5% CO₂, saturated moisture, at 37° C. The cell viability inhibition effects of lovastatin and the Monascus composition were compared with the same method as the above lung cancer cell lines. The result as shown in FIG. 8 also shows the Monascus composition having a higher inhibition effect than the lovastatin.

It is obvious the anti-cancer activity of Monascus compositions is superior to lovastatin based on the above in vivo and in vitro tests. The data above also shows that the Monascus compositions containing lovastatin were more effective than lovastatin alone for treatment and prevention of cancer.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art) Therefore, the scope of the appended claims should be accorded the broadest interpretation so as to encompass all such modifications and similar arrangements.

What is claimed is:

1. A composition of Monascus extract with at least one component with three characteristic ultraviolet absorption peaks at 230, 237 and 246 nm.

2. The composition as claimed in claim 1, wherein the Monascus extract comprises a component having an identifying characteristic of a retention time of 7.7 min. on HPLC using a Cosmosil 5C18-ARII column (4.6.times.250mm) and elution with methanol-water-H₃PO₄ (3.2 L:0.8 L:1.6 ml) at a flow rate of 1 ml/min and detected with 237 nm.

3. The composition as claimed in claim 2, wherein the Monascus extract comprises two components having identifying characteristics of retention time of 6.6 and 8.7 min. respectively on HPLC using a Cosmosil 5C18-ARII column (4.6.times.250mm) and elution with methanol-water-H₃PO₄ (3.2 L: 0.8 L: 1.6 ml) at a flow rate of 1 ml/min and detected with 237 nm.

4. The composition as claimed in claim 1, wherein the Monascus extract comprises two components having identifying characteristics of retention time of 7.7 and 8.7 min. respectively on HPLC using a Cosmosil 5C18-ARII column (4.6.times.250mm) and elution with methanol-water-H₃PO₄ (3.2 L: 0.8 L: 1.6 ml) at a flow rate of 1 ml/min and detected with 237 nm.

5. The composition as claimed in claim 4, wherein the Monascus extract comprises two components having identifying characteristics of retention time of 9.9 and 12 min. respectively on HPLC using a Cosmosil 5C18-ARII column (4.6.times.250 mm) and elution with methanol-water-H₃PO₄ (3.2 L:0.8 L:1.6 ml) at a flow rate of 1 ml/min and detected with 237 nm.

6. The composition as claimed in claim 1, wherein the Monascus extract comprises lovastatin.

7. The composition as claimed in claim 1, wherein the Monascus extract is a fermentation product of Monascus purpureus Went, Monascus ruber van Tieghem, Monascus pilosus Sato, Monascus albidus Sato Monascus pubigerus Sato, Monascus floridanus, Monascus sanguineus, Monascus kaoliang or Monascus albus.

8. The composition as claimed in claim 1, wherein the Monascus extract is a fermentation product of strain ATCC No. 16360, ATCC No. 16363, ATCC No. 16364, ATCC No. 16368, ATCC No. IFO4520, ATCC No. 22080, ATCC No. 16385, ATCC No. 16427, ATCC No. 15670, ATCC No. 16246, ATCC No.18199, ATCC No. 36928, ATCC No. 16365, CCRC No. 31499, CCRC No. 31502, CCRC No. 31503, CCRC No. 31505, CCRC No. 31526, CCRC No. 31527, CCRC No. 31529, CCRC No. 31530, CCRC No. 31532, CCRC No. 31533, CCRC No. 31535, CCRC No. 31540, or CCRC No. 31542.

9. A pharmaceutical composition for treatment and prevention of cancer, comprising:
a Monascus composition, comprising at least one component with three characteristic ultraviolet absorption peaks at 230, 237 and 246 nm; and
a pharmaceutically acceptable carrier or excipient.

10. The pharmaceutical composition as claimed in claim 9, wherein the Monascus composition comprises a component having an identifying characteristic of a retention time of 7.7 min. on HPLC using a Cosmosil 5C18-ARII column (4.6.times.250 mm) and elution with methanol-water-H₃PO₄ (3.2 L:0.8 L:1.6 ml) at a flow rate of 1 ml/min and detected with 237 nm.

11. The pharmaceutical composition as claimed in claim 10, wherein the Monascus composition comprises two components having identifying characteristics of retention time of 6.6 and 8.7 min. respectively on HPLC using a Cosmosil 5C18-ARII column (4.6.times.250mm) and elution with methanol-water-H₃PO₄ (3.2 L:0.8 L:1.6 ml) at a flow rate of 1 ml/min and detected with 237 nm.

12. The pharmaceutical composition as claimed in claim 11, wherein the Monascus composition comprises two components having identifying characteristics of retention time of 9.9 and 12 min. respectively on HPLC using a Cosmosil 5C18-ARII column (4.6.times.250 mm) and elution with methanol-water-H₃PO₄ (3.2 L:0.8 L:1.6 ml) at a flow rate of 1 ml/min and detected with 237 nm.

13. The pharmaceutical composition as claimed in claim 9, wherein the Monascus composition comprises lovastatin.

14. The pharmaceutical composition as claimed in claim 9, wherein the Monascus composition is an fermentation product of Monascus purpureus Went, Monascus ruber van Tieghem, Monascus pilosus Sato, Monascus albidus Sato Monascus pubigerus Sato, Monascus floridanus, Monascus sanguineus, Monascus kaoliang or Monascus albus.

15. The pharmaceutical composition as claimed in claim 9, wherein the Monascus composition is an fermentation product of strain ATCC No. 16360, ATCC No. 16363, ATCC No. 16364, ATCC No. 16368, ATCC No. IF04520, ATCC No. 22080, ATCC No. 16385, ATCC No. 16427, ATCC No. 15670, ATCC No. 16246, ATCC No.18199, ATCC No. 36928, ATCC No. 16365, CCRC No. 31499, CCRC No. 31502, CCRC No. 31503, CCRC No. 31505, CCRC No. 31526, CCRC No. 31527, CCRC No. 31529, CCRC No. 31530, CCRC No. 31532, CCRC No. 31533, CCRC No. 31535, CCRC No. 31540, or CCRC No. 31542.

16. The pharmaceutical composition as claimed in claim 9, wherein the Monascus composition is effective for treating lung cancer or breast cancer.

17. The pharmaceutical composition as claimed in claim 9, wherein the Monascus composition is a solid fermentation product of rice fermented with a Monascus strain.

18. The pharmaceutical composition as claimed in claim 9, wherein the Monascus composition is a liquid fermentation product of a Monascus strain.

19. A method for producing a Monascus composition as claimed in claim 9, the method comprising:
providing a liquid culture medium of grain/starch:peptone:MgSO₄.7H₂O:water=1-7 g: 0.5-3 g: 0.1-1 g: 100 g;
culturing a Monascus strain in the liquid culture medium at a temperature of about 20 to 35° C., an agitation rate of 100 to 400 rpm, and an aeration rate of 0.1 to 1 vvm for 4 to about 12 days; and
filtering the liquid culture medium to obtain a Monascus extract.

20. The method as claimed in claim 19, wherein the liquid culture medium further comprises glycerin in a concentration of 10 ml or below.

21. The method as claimed in claim 19, wherein the liquid culture medium further comprises glucose in a concentration of 6 g or below.

22. The method as claimed in claim 19, wherein the liquid culture medium further comprises a malt extract in a concentration of 3 g or below.

23. The method as claimed in claim 19, wherein the liquid culture medium further comprises casein in a concentration of 5 g or below.

24. The method as claimed in claim 19, wherein the Monascus strain is cultured at about 25° C.

25. The method as claimed in claim 19, wherein the Monascus strain is cultured at 0.5 vvm aeration rate.

26. The method as claimed in claim 19, wherein the Monascus strain is cultured for about 14 days.

27. The method as claimed in claim 19, wherein the Monascus strain is Monascus purpureus Went Monascus ruber van Tieghem Monascus pilosus Sato Monascus albidus Sato Monascus pubigerus Sato Monascus floridanus Monascus sanguineus Monascus kaoliang or Monascus albus.

28. The method as claimed in claim 19, wherein the Monascus strain is strain ATCC No. 16360, ATCC No. 16363, ATCC No. 16364, ATCC No. 16368, ATCC No. IF04520, ATCC No. 22080, ATCC No. 16385, ATCC No. 16427, ATCC No. 15670, ATCC No. 16246, ATCC No.18199, ATCC No. 36928, ATCC No. 16365, CCRC No. 31499, CCRC No. 31502, CCRC No. 31503, CCRC No. 31505, CCRC No. 31526, CCRC No. 31527, CCRC No. 31529, CCRC No. 31530, CCRC No. 31532, CCRC No. 31533, CCRC No. 31535, CCRC No. 31540, or CCRC No. 31542.

29. A method for preventing or treating cancer in a mammal which comprises administrating to the mammal a sufficient amount of a Monascus composition.

30. The method as claimed in claim 9, wherein the cancer is lung cancer or breast cancer.

31. The method as claimed in claim 9, wherein the Monascus composition comprises at least one component with three characteristic ultraviolet absorption peaks at 230, 237 and 246 nm.

32. The method as claimed in claim 31, wherein the Monascus composition comprises a component having an identifying characteristic of a retention time of 7.7 min. on HPLC using a Cosmosil 5C18-ARII column (4.6.times.250mm) and elution with methanol-water-H₃PO₄ (3.2 L:0.8 L:1.6 ml) at a flow rate of 1 ml/min and detected with 237 nm.

33. The method as claimed in claim 32, wherein the Monascus composition is an fermentation product of Monascus purpureus Went, Monascus ruber van Tieghem, Monascus pilosus Sato, Monascus albidus Sato Monascus pubigerus Sato, Monascus floridanus, Monascus sanguineus, Monascus kaoliang or Monascus albus.
